(19) United States
(12) Patent Application Publication
(10) Pub. No.: US 2014/0329774 A1
(43) Pub. Date: Nov. 6, 2014

(54) INJECTABLE COMPOSITION CONTAINING PHOSPHATIDYLCHOLINE DEVOID OF SODIUM DEOXYCHOLATE AND PREPARING METHOD THEREOF

(71) Applicant: AMI Pharm Co., Lid., Seoul (KR)

(72) Inventors: Ki Teak LEE, Seongnam-si (KR); Jong Hyuk LEE, Seoul (KR); IIkyeong SEONG, Cheongju-si (KR)

(21) Appl. No.: 14/334,348

(22) Filed: Jul. 17, 2014

Related U.S. Application Data
(63) Continuation of application No. PCT/KR2013/000999, filed on Feb. 7, 2013.

(30) Foreign Application Priority Data
Feb. 7, 2012 (KR) 10-2012-0012360

(51) Int. Cl. A61K 9/08; A61K 9/00; A61K 47/10; A61K 31/685

(52) U.S. Cl. CPC A61K 9/08; A61K 31/685; A61K 9/0019; A61K 47/10
USPC 514/114

(57) ABSTRACT

The present invention includes an injectable composition containing phosphatidylcholine including phosphatidylcholine; ethanol; propylene glycol and/or benzyl alcohol; polysorbate and/or macrogol 15 hydroxystearate; and a balance of water or water for injection, and a preparing method thereof. An injectable composition of the present invention includes no sodium deoxycholate, which is carcinogenic, and therefore a safer injectable composition of phosphatidylcholine can be prepared.

### CROSS-REFERENCE TO RELATED APPLICATIONS

This application is a continuation of International Application No. PCT/KR2013/000999, filed on Feb. 7, 2013, and claims priority from and the benefit of Korean Patent Application No. 10-2012-0012360, filed on Feb. 7, 2012, each of which is hereby incorporated by reference for all purposes as if fully set forth herein.

### BACKGROUND

1. Field

The present invention relates to an injectable composition containing phosphatidylcholine devoid of sodium deoxycholate and a preparing method thereof, and more particularly to an injectable composition containing phosphatidylcholine comprising phosphatidylcholine; ethanol; propylene glycol and/or benzyl alcohol; polysorbate and/or macrogol 15 hydroxystearate; and a balance of water or water for injection, and to a preparing method thereof.

2. Discussion of the Background

Phosphatidylcholines are a class of phospholipids that contain choline as a head group. They are widely present in animals, plants, yeasts and fungi, and are also known as lecithin. They are the membrane phospholipids of mammals and are found mainly in brains, nerves, blood cells, egg yolks and the like. In plants, phosphatidylcholines are found in soybeans, sunflower seeds, wheat germs and the like. Phosphatidylcholines generally contain saturated fatty acid at position 1 and unsaturated fatty acid at position 2 of glycerol.

Normally, liver cells synthesize phospholipids as required, but if liver cells are damaged, they cannot synthesize an increased amount of phospholipids required to restore the membrane structures within a short time.

Generally, when the synthesis of albumin and coagulation factors decreases, a damaged liver has a significantly reduced ability to synthesize phospholipids, and a significant amount of energy is consumed to produce new phospholipids.

The loss of phospholipids by liver disease causes damage to liver cell membranes and organelles, which is difficult to restore. In an attempt to prevent this loss, there was developed a method in which high purity phosphatidylcholine is supplied into the body so that it is bound to the membrane structure of damaged liver cells to restore the membrane. When phosphatidylcholine is supplied, the exchange of nutrients and electrolytes across membranes is increased, the activity of phospholipid-dependent enzymes is also increased, and high-energy phosphatidylcholine molecules are bound to liver cells to reduce the burden to supply a large amount of energy required for the production of structural and functional is components of membrane systems to the liver. Based on the above facts, injectable formulations containing high-purity phosphatidylcholine have been developed and used for recovery from hepatic coma caused by liver cirrhosis.

In addition, since it was recently reported that phosphatidylcholine has the effect of decomposing locally accumulated fat, injectable formulations containing phosphatidylcholine have been widely used for local lipolysis for beauty purposes.

Drugs need to be solubilized before injection. If drugs are injected in a non-solubilized state, they are not easily decomposed into single molecules, and the desired levels thereof in blood cannot be obtained. In addition, because non-solubilized drugs can block blood vessels to cause thrombosis, they are not used as injectable formulations. If drugs form a suspended precipitate without being solubilized when they are injected intravenously, large particles will block blood vessels and affect blood flow in tissue around the blocked blood vessels or damage or stimulate the tissue to cause itching, pain, redness, etc. In severe cases, embolism may also occur.

Phosphatidylcholine is a phospholipid component that is not easily soluble in water-soluble solvents for injection. Thus, sodium deoxycholate is added in order to solubilize phosphatidylcholine.

However, sodium deoxycholate that is the main component of bile acid can cause safety concerns when it is applied to body tissues other than the small intestines. In addition, it may cause colorectal cancer. Thus, it appears that sodium deoxycholate is unsuitable for use as the active ingredient of a solubilizer for a drug for intravenous (or subcutaneous) injection.

Accordingly, there is an urgent need for the development of a phosphatidylcholine-containing injectable composition which contains no sodium deoxycholate and in which phosphatidylcholine is solubilized so as to be injectable intravenously (or subcutaneously).

### SUMMARY

Accordingly, the present inventors have conducted studies on an injectable composition which comprises no sodium deoxycholate and in which phosphatidylcholine is stably solubilized. As a result, the present inventors have found that phosphatidylcholine is solubilized by a combination of some solubilizers and solubilization aids without sodium deoxycholate, thereby completing the present invention.

Therefore, it is an object of the present invention to provide an injectable composition containing phosphatidylcholine including
(a) 2-10% (w/v) of phosphatidylcholine
(b) 5-40% (w/v) of ethanol
(c) 2-20% (w/v) of one or more selected from the group consisting of propylene glycol and benzyl alcohol
(d) 1-30% (w/v) of one or more selected from the group including polysorbate and macrogol 15 hydroxystearate, and
(e) a balance of water or water for injection.

Another object of the present invention is to provide a method for preparing an injectable composition containing phosphatidylcholine including the steps of:
(a) adding phosphatidylcholine and one or more selected from the group consisting of polysorbate and macrogol 15 hydroxystearate to ethanol;
(b) adding one or more selected from the group consisting of propylene glycol and benzyl alcohol to the mixture of step (a); and
(c) adding water or water for injection to the mixture of step (b) until a predetermined total volume is reached.

To achieve the above object, the present invention provides an injectable composition containing phosphatidylcholine, including:
(a) 2-10% (w/v) of phosphatidylcholine,
(b) 5-40% (w/v) of ethanol,
(c) 2-20% (w/v) of one or more selected from the group consisting of propylene glycol and benzyl alcohol,
(d) 1-30% (w/v) of one or more selected from the group including polysorbate and macrogol 15 hydroxystearate, and
(e) a balance of water or water for injection.

To achieve another object, the present invention provides a method for preparing an injectable composition containing phosphatidylcholine including the steps of:
(a) adding phosphatidylcholine and one or more selected from the group consisting of polysorbate and macrogol 15 hydroxystearate to ethanol;
(b) adding one or more selected from the group consisting of propylene glycol and benzyl alcohol to the mixture of step (a); and
(c) adding water or water for injection to the mixture of step (b) until a predetermined total volume is reached.

It is to be understood that both the foregoing general description and the following detailed description are exemplary and explanatory and are intended to provide further explanation of the invention as claimed.

### BRIEF DESCRIPTION OF DRAWINGS

The accompanying drawings, which are included to provide a further understanding of the invention and are incorporated in and constitute a part of this specification, illustrate embodiments of the invention, and together with the description serve to explain the principles of the invention.

FIG. 1 shows comparison of the status of compositions for solubilization of phosphatidylcholine and the numbers in vial refers the test numbers of the Table 2 of the Example 2.

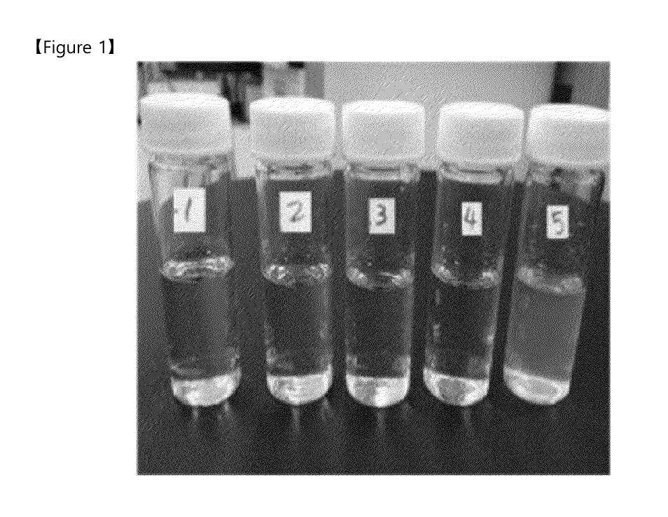

### DETAILED DESCRIPTION OF THE ILLUSTRATED EMBODIMENTS

Hereinafter, exemplary embodiments of the present invention will be described in detail.

An injectable composition of the present invention is characterized by including:
(a) 2-10% (w/v) of phosphatidylcholine,
(b) 5-40% (w/v) of ethanol,
(c) 2-20% (w/v) of one or more selected from the group consisting of propylene glycol and benzyl alcohol,
(d) 1-30% (w/v) of one or more selected from the group consisting of polysorbate and macrogol 15 hydroxystearate and
(e) a balance of water or water for injection.

An injectable formulation is obtained by dissolving an active ingredient and other additives in distilled water for injection, filtering the solution through a bacterial filter to remove bacterial cells, and filling the filtered solution into a vial in an aseptic condition, and then sealing the vial. Phosphatidylcholine that is contained in the injectable composition according to the present invention is also known as lecithin and is the most typical phospholipid. It accounts for about 70% of total phospholipids in yolk eggs and about 60% of total phospholipids in human serum.

Soybean lecithin contains a component consisting of two fatty acids and linoleic acid, unlike other lecithins, and thus has the effect of improving lipid metabolism. In the composition of the present invention, the concentration of phosphatidylcholine is 2-10% (w/v).

In addition, ethanol that is contained in the injectable composition according to the present invention is ethane with a hydrogen molecule replaced by a hydroxyl radical. The concentration of ethanol in the composition according to the present invention is 5-40% (w/v).

In addition, propylene glycol that is contained in the injectable composition according to the present invention is a colorless transparent liquid similar to glycerin and is generally used as a preservative, because it has moisture-absorbing and moisture-holding properties and preservative properties. Moreover, benzyl alcohol that is contained in the injectable composition according to the present invention is one of aromatic alcohols, which is a colorless transparent liquid. It has a peculiar fragrance and a sharp taste and is generally used as a dissolution agent, an extraction agent, a volatilization inhibitor, a food spice and the like. The concentration of propylene glycol and/or benzyl alcohol in the composition according to the present invention is 2-20% (w/v).

In addition, macrogol 15 hydroxystearate that is contained in the injectable composition according to the present invention is generally used as a nonionic surfactant, has good chemical stability and low toxicity and easily dissolves in water, ethanol and 2-propanol. Further, polysorbate that is contained in the injectable composition according to the present invention is a polyoxyethylene higher aliphatic alcohol consisting of ethylene oxide bonded to sorbitan fatty acid ester and is a kind of nonionic surfactant. It is divided, according to the number of polyoxyethylene groups and the kind of fatty acid, into polysorbate 20 (monolauric acid), 40 (monopalmitic acid), 60 (monostearic acid), 65 (tristearic acid) and 80 (mono-oleic acid). Among them, polysorbate 80 may be used in the present invention. The concentration of polysorbate and/or macrogol in the composition according to the present invention is 1-30% (w/v).

In addition, the water for injection that is contained in the injectable composition according to the present invention is distilled water made to dissolve a solid formulation or dilute a water-soluble formulation. Specific examples thereof include glucose injection, xylitol injection, D-mannitol injection, fructose injection, physiological saline, dextran 40 injection, dextran 70 injection, amino acid injection, Ringer solution, lactic acid-Ringer solution or the like.

The injectable composition of the present invention is a phosphatidylcholine-containing injectable composition containing no sodium deoxycholate and does not cause the risk of colorectal cancer.

The above-described inventive phosphatidylcholine-containing injectable composition, which comprises phosphatidylcholine, ethanol, propylene glycol and/or benzyl alcohol, polysorbate and/or macrogol 15 hydroxystearate, and a balance of water or water for injection, has not been reported before the present invention.

A method of the present invention is characterized by comprising the steps of:
(a) adding phosphatidylcholine and one or more selected from the group consisting of polysorbate and macrogol 15 hydroxystearate to ethanol;
(b) adding one or more selected from the group consisting of propylene glycol and benzyl alcohol to the mixture of step (a); and
(c) adding water or water for injection to the mixture of step (b) until a predetermined total volume is reached.

The preparing method of the injectable composition could be explain step by step as follows:
(a) step: adding (and mixing (suspending or blending)) phosphatidylcholine and one or more selected from the group consisting of polysorbate and macrogol 15 hydroxystearate to ethanol.

The ingredients of phosphatidylcholine, polysorbate and macrogol 15 hydroxystearate in step (a) are same as the above-mentioned. After mixing the ingredients of step (a), the mixture is stirred until a clear solution is formed.

The (a) step may comprise adding 2-10% (w/v) of phosphatidylcholine and 2-20% (w/v) of one or more selected from the group consisting of polysorbate and macrogol 15 hydroxystearate to 5-40% (w/v) of ethanol.

(b) step: adding (and mixing (suspending or blending)) one or more selected from the group consisting of propylene glycol and benzyl alcohol to the mixture of step (a)

The ingredients of propylene glycol and benzyl alcohol, and water for injection in step (b) are same as the above-mentioned.

The (b) step may comprise adding 1-30% (w/v) of one or more selected from the group consisting of propylene glycol and benzyl alcohol to the mixture of step (a).

(c) step: adding (and mixing (suspending or blending)) water or water for injection to the mixture of step (b) until a predetermined total volume is reached

After adding water or water for injection of step (c), the resulting solution is homogenized.

In one example of the present invention, in order to find an injectable composition capable of solubilizing phosphatidylcholine without having to use sodium deoxycholate, unlike conventional injectable compositions containing phosphatidylcholine, the abilities of various combinations of additives for intravenous injection to solubilize phospholipids were evaluated.

As a result, it was shown that one or more selected from the group consisting of polysorbate and macrogol 15 hydroxystearate and one or more selected from the group consisting of propylene glycol and benzyl alcohol are suitable as solubilizers (see Example 1).

In another example of the present invention, using the composition found to be the most excellent combination in the above-described example (Example 1), the degree of solubilization was measured while changing the concentrations of the components of the composition.

As a result, it was shown that other solubilizers (PEG 400, urea, β-cyclodextrin, sorbitol, span 80, poloxamer 188, glycerol, etc.) have no significant effect.

Accordingly, the injectable composition of phosphatidylcholine, which comprises phosphatidylcholine, ethanol, propylene glycol and/or benzyl alcohol, polysorbate and/or macrogol 15 hydroxystearate, and a balance of water or water for injection, can be used as an injectable composition having an excellent ability to solubilize phosphatidylcholine without having to use sodium deoxycholate.

The method for administration of the injectable composition of the present invention is not specifically limited, but can be suitably selected in view of the severity of disease, and the patient's age, sex and other conditions. The route for administration of the injectable composition of the present invention is not specifically limited, but may be subcutaneous injection, transdermal injection, intravenous injection, intramuscular injection, intraperitoneal injection or the like.

Accordingly, the present invention provides an injectable composition of phosphatidylcholine comprising phosphatidylcholine; ethanol; propylene glycol and/or benzyl alcohol; polysorbate and/or macrogol 15 hydroxystearate; and a balance of water or water for injection. An injectable composition of the present invention comprises no sodium deoxycholate and phosphatidylcholine is stably solubilized in thereof. As a result, the present invention provides more safe injectable composition of phosphatidylcholine.

Hereinafter, the present invention will be described in detail with reference to following Examples.

However, the following Examples are only for illustrative purposes and are not intended to limit the scope of the invention.

### Example 1

### Experiment on Solubilization of Phosphatidylcholine Using Various Components of Composition for Solubilization

In order to find an injectable composition capable of solubilizing phosphatidylcholine without having to use sodium deoxycholate, unlike conventional injectable compositions containing phosphatidylcholine, the abilities of various combinations of additives for intravenous injection to solubilize phospholipids were evaluated.

Phosphatidylcholine, polysorbate 80 and ethanol were mixed with each other in the amounts shown in Table 1 below, and the mixture was stirred at 300 rpm at 30° C. for 30 minutes in a closed space under light-free conditions.

The phosphatidylcholine was purchased from Lipoid GmbH (Germany) (Cat. No. 368202, Model: PHOSPHOLIPON® 90G).

To the mixture, 45 mg of benzyl alcohol was added, and other various solubilizers were added. Water for injection was added thereto until a total volume of 5 ml was reached. The resulting solution was stirred at 300 rpm at 30° C. for 3 hours in a closed space under light-free conditions, thereby preparing an injectable composition.

Visual observation was carried out to determine whether the above-prepared injectable composition undergoes phenomena, including precipitation, suspension and phase separation, and is transparent. As a control, a conventional injectable formulation containing sodium deoxycholate (5 mg injectable formulation comprising 250 mg phosphatidylcholine, 120 mg sodium deoxycholate, 12 mg sodium chloride, 45 mg benzyl alcohol, 10 mg ethanol and a balance of water for injection) was used.

Observation results were expressed as follows: (+): there are no phenomena, including precipitation, suspension and phase separation, and transparency is equal to or higher than that of the control; and (−): solubilization is insufficient, or transparency is lower than that of the control group.

As a result, as can be seen in Table 1 below, when PEG 400, urea, β-cyclodextrin, sorbitol, span 80, poloxamer 188 and glycerol were added, solubilization was insufficient, and when propylene glycol or macrogol 15 hydroxystearate was added as an additive, the degree of solubilization was similar to that of the control, and the composition was transparent. Thus, it was determined that a combination of phosphatidylcholine, ethanol, benzyl alcohol, polysorbate, propylene glycol or macrogol 15 hydroxystearate, and water for injection can be used as a phosphatidylcholine-containing injectable composition free of sodium deoxycholate.

TABLE 1
Solubility according to additives

| No. | 1 | 2 | 3 | 4 | 5 |
|---|---|---|---|---|---|
| essential | 250 mg | 250 mg | 250 mg | 250 mg | 250 mg |
| phospholipid | | | | | |
| material | | | | | |
| polysorbate | 500 mg | 500 mg | 500 mg | 500 mg | 500 mg |
| 80 | | | | | |
| ethanol | 500 mg | 500 mg | 500 mg | 500 mg | 500 mg |
| benzyl | 45 mg | 45 mg | 45 mg | 45 mg | 45 mg |
| alcohol | | | | | |
| other | propylene | PEG 400 | urea | β-cyclo- | sorbitol |
| additives | glycol | 500 mg | 500 mg | dextrin | 500 mg |
| | 500 mg | | | 100 mg | |
| water for | remainder | remainder | remainder | remainder | remainder |
| injection | | | | | |
| status | + | − | − | − | − |

| No. | 6 | 7 | 8 | 9 |
|---|---|---|---|---|
| essential | 250 mg | 250 mg | 250 mg | 250 mg |
| phospholipid | | | | |
| material | | | | |
| polysorbate | 500 mg | 500 mg | 500 mg | 500 mg |
| 80 | | | | |
| ethanol | 500 mg | 500 mg | 500 mg | 500 mg |
| benzyl | 45 mg | 45 mg | 45 mg | 45 mg |
| alcohol | | | | |
| other | Span 80 | poloxamer | glycerol | Macrogol 15 |
| additives | 500 mg | 188 | 500 mg | Hydroxy- |
| | | 100 mg | | stearate |
| Water for | remainder | remainder | remainder | remainder |
| injection | | | | |
| status | − | − | − | + |

### Example 2

### Experiment on Solubilization of Phosphatidylcholine Using Various Concentrations of Components of Composition for Solubilization

The components selected in Example 1 were combined with each other so as to have various components and concentrations, and the conditions in which an optimal injectable composition for solubilization is obtained were determined.

Phosphatidylcholine, polysorbate 80 and/or macrogol 15 hydroxystearate, and ethanol were mixed with each other in the amounts shown in Table 2 below, and the mixture was stirred at 300 rpm at 30° C. or 30 minutes in a closed space under light-free conditions.

To the mixture, propylene glycol and/or benzyl alcohol was added in the amounts shown in Table 2 below, and water for injection was added thereto until a total volume of 5 ml was reached. The resulting solution was stirred at 300 rpm at 30° C. for 3 hours in a closed space under light-free conditions, thereby preparing an injectable composition.

Visual observation was carried out to determine whether the above-prepared injectable composition undergoes phenomena, including precipitation, suspension and phase separation, and is transparent. As a control, a conventional injectable formulation containing sodium deoxycholate (5 mg injectable formulation comprising 250 mg phosphatidylcholine, 120 mg sodium deoxycholate, 12 mg sodium chloride, 45 mg benzyl alcohol, 10 mg ethanol and a balance of water for injection) was used.

Observation results were expressed as follows: (+): there are no phenomena, including precipitation, suspension and phase separation, and transparency is equal to or higher than that of the control; and (−): solubilization is insufficient, or transparency is lower than that of the control group.

As a result, as can be seen in Table 2 below, it was found that, when propylene glycoland benzyl alcohol were not added, the solubilizing effect was insufficient, and when propylene glycol and/or benzyl alcohol, and polysorbate and/or macrogol 15 hydroxystearatewere added as additives, the degree of solubilization was sufficient, similar to that of the control, and the composition was transparent. Thus, it determined found that a combination of phosphatidylcholine, ethanol, propylene glycol and/or benzyl alcohol, and polysorbate and/or macrogol 15 hydroxystearate can be used as a phosphatidylcholine-containing injectable composition free of sodium deoxycholate.

TABLE 2
Solubility according to the concentration of additives

| Test No. | 1 | | 2 | | 3 | | 4 | | 5 | |
|---|---|---|---|---|---|---|---|---|---|---|
| essential | 287.5 | mg | 287.5 | mg | 287.5 | mg | 287.5 | mg | 287.5 | mg |
| phospholipid | | | | | | | | | | |
| material | | | | | | | | | | |
| propylene- | | | | | 500 | mg | 500 | mg | | |
| glycol | | | | | | | | | | |
| benzyl | 200 | mg | 100 | mg | | | | | | |
| alcohol | | | | | | | | | | |
| Polysorbate | 120 | mg | 200 | mg | | | 600 | mg | 600 | mg |
| 80(Tween80) | | | | | | | | | | |
| Ethanol | 1500 | mg | 1500 | mg | 1000 | mg | 500 | mg | 500 | mg |
| Macrogol 15 | | | | | 500 | mg | 500 | mg | 500 | mg |
| Hydroxy- | | | | | | | | | | |
| stearate | | | | | | | | | | |
| Water for | remainder | | remainder | | remainder | | remainder | | remainder | |
| injection | | | | | | | | | | |
| Total | 5 | ml | 5 | ml | 5 | ml | 5 | ml | 5 | ml |

| | | | | | |
|---|---|---|---|---|---|
| status | + | + | + | + | − |

What is claimed is:

1. An injectable composition containing phosphatidylcholine free of sodium deoxycholate, comprising:
2-10% (w/v) of phosphatidylcholine;
5-40% (w/v) of ethanol;
2-20% (w/v) of one or more selected from the group consisting of propylene glycol and benzyl alcohol;
1-30% (w/v) of one or more selected from the group consisting of polysorbate and macrogol 15 hydroxystearate; and
a balance of water or water for injection.

2. A method for preparing an injectable composition containing phosphatidylcholine, the method comprising:
(a) adding phosphatidylcholine and one or more selected from the group consisting of polysorbate and macrogol 15 hydroxystearate to ethanol;
(b) adding one or more selected from the group consisting of propylene glycol and benzyl alcohol to the mixture of step (a); and
(c) adding water or water for injection to the mixture of step (b) until a predetermined total volume is reached.

3. The method of claim 2, the (a) step comprising:
adding 2-10% (w/v) of phosphatidylcholine and 2-20% (w/v) of one or more selected from the group consisting of polysorbate and macrogol 15 hydroxystearate to 5-40% (w/v) of ethanol.

4. The method of claim 2, the (b) step comprising:
adding 1-30% (w/v) of one or more selected from the group consisting of propylene glycol and benzyl alcohol to the mixture of step (a).

5. An injectable composition containing phosphatidylcholine free of sodium deoxycholate, consisting of:
2-10% (w/v) of phosphatidylcholine;
5-40% (w/v) of ethanol;
2-20% (w/v) of one or more selected from the group consisting of propylene glycol and benzyl alcohol;
1-30% (w/v) of one or more selected from the group consisting of polysorbate and macrogol 15 hydroxystearate; and
a balance of water or water for injection.
